(19) United States
(12) Patent Application Publication
(10) Pub. No.: US 2008/0306442 A1
(43) Pub. Date: Dec. 11, 2008

(54) INTRODUCER SHEATH

(75) Inventors: Earl Bardsley, Newton, MA (US); Jianlu Ma, Maple Grove, MN (US); Thomas J. McEvoy, Minnetonka, MN (US)

Correspondence Address: HAMRE, SCHUMANN, MUELLER & LARSON, P.C., P.O. BOX 2902, MINNEAPOLIS, MN, 55402-0902, US

(73) Assignee: EPITEK, INC., Bloomington, MN (US)

(21) Appl. No.: 12/119,026

(22) Filed: May 12, 2008

Related U.S. Application Data
(60) Provisional application No. 60/938,636, filed on May 17, 2007.

(51) Int. Cl. A61M 5/32; A61M 29/00

(52) U.S. Cl. 604/104; 604/164.04; 604/164.01

(57) ABSTRACT

An introducer sheath includes an elongated longitudinal body having opposite ends longitudinally disposed. An opening is present at each end of the elongated body and a channel extends longitudinally through the openings of the elongated body. The elongated body of the introducer sheath includes a varying stiffness from one end to the other end. As one example, an introducer sheath can introduce devices and/or medical treatment products used in minimally invasive medical procedures.

### CROSS-REFERENCE TO RELATED APPLICATIONS

The present application claims the benefit of U.S. Provisional Patent Application Ser. No. 60/938,636 entitled “INTRODUCER SHEATH,” filed on May 17, 2007, which is herewith incorporated by reference in its entirety.

### FIELD

An introducer sheath is described, which can provide minimally invasive access into the body of a patient, and includes a variable stiffness along its longitudinal profile. For example, an introducer sheath is provided that is particularly helpful for introducing devices and/or medical treatment products in minimally invasive medical procedures.

### BACKGROUND

Introducer sheaths are well known and widely used, such as for introducing tools to be used in certain medical procedures and applications. Such introducer sheaths have been employed for procedures that are aimed at being minimally invasive and avoiding trauma to a patient.

For example, introducer sheaths are commonly used to feed catheter products into arteries and veins, and more generally into a patient's body. These sheaths typically are constructed of materials having a constant durometer, which have no varying flexibility or stiffness. Rather, the constant durometer of the material provides a sheath with the same overall stiffness (or flexibility) that does not vary along its longitudinal profile.

However, such sheaths have shortcomings in that they are not both adequately stiff (i.e. too flexible) to push the sheath to a location inside a patient, while also being adequately flexible (i.e. too stiff) to articulate over soft tissue and/or organs once inside a patient.

Thus, improvements may yet be made to introducer sheaths. For example, there is a need to provide an introducer sheath with a varying flexibility along its length, and to provide for example, an improvement for securing the sheath during its use while maintaining a minimally invasive quality of the sheath.

### SUMMARY

The following disclosure provides an improved introducer sheath. An introducer sheath as will be described herein includes a variable stiffness along its longitudinal profile, and provides a minimally invasive access into a location of the body of a patient.

As one example, an introducer sheath as described herein can be employed for introducing devices and/or medical treatment products used in minimally invasive medical procedures. In one particular example, the introducer sheath described herein can be useful for introducing such devices that employ a sub-xiphoid access in procedures performed on the heart, for instance a left atrial appendage closure device.

In one embodiment, an introducer sheath includes an elongated longitudinal body having opposite ends longitudinally disposed. An opening is present at each end, such that the openings define a channel extending longitudinally through the elongated body. The elongated body of the introducer sheath includes a wall about the channel having a varying stiffness from one end to the other end.

In one embodiment, an introducer sheath includes a proximate end and a distal end. The proximate end is operative for pushing the distal end and precedes the distal end, in which the distal end is insertable to a certain location in the body of a patient. The introducer sheath includes a decreasing stiffness from the proximate end to the distal end along the elongated body, such that the distal end is more flexible than the proximate end.

In yet another embodiment, an introducer sheath includes a proximate end and a distal end. The distal end includes a tip configured to allow for ease of introduction and movement of the introducer sheath in the body of a patient.

In another embodiment, an introducer sheath includes a proximate end and a distal end. The distal end includes an expander. The expander is operable for expanding a space inside the body of a patient, so as to provide a working space to allow easier performance of a procedure inside the body.

In yet another embodiment, an introducer sheath includes a retention mechanism. The retention mechanism is operable for retaining the introducer sheath in place once the introducer sheath is positioned, such as after a portion of the introducer sheath has been inserted into the body of a patient at a desired location. In one embodiment, the retention mechanism is adjustable, such that the introducer sheath can be inserted and secured in the body of a patient at varying lengths.

In another embodiment, an introducer sheath includes a device lock. The device lock is operable for securing a device inserted through the elongated body of the introducer sheath, and which is to be used for a procedure inside the body of a patient.

The above and other various features of novelty and advantages are pointed out herein. For better understanding of the technical disclosure, reference should also be made to the drawings, which form a further part hereof, and to the accompanying descriptive matter, in which some embodiments of the technical concepts and their advantages are illustrated and described.

### BRIEF DESCRIPTION OF THE DRAWINGS

The embodiments illustrated in the drawings are exemplary only, and are in accordance with inventive concepts of this technical disclosure.

FIG. 1 is a partial perspective view of one embodiment of an introducer sheath.

FIG. 2 is a perspective view of another embodiment of an introducer sheath.

FIG. 3 is a perspective view of another embodiment of an introducer sheath.

FIG. 4 is a perspective view of yet another embodiment of an introducer sheath.

FIG. 5 is a perspective view of yet another embodiment of an introducer sheath.

FIG. 6 is a side view of another embodiment of an introducer sheath.

FIG. 7 is a partial side sectional view of one embodiment of a distal end of an introducer sheath.

FIG. 8 is a sectional view of another embodiment of an introducer sheath.

FIG. 9 is a view of a chest cavity showing one embodiment of where an introducer sheath can be used as an access.

FIG. 10 is an anterior view of a heart showing the left atrial appendage as one embodiment of a tissue targeted for access by an introducer sheath.

FIG. 11A is a side view of one embodiment of a device lock for use with an elongated body of an introducer sheath.

FIG. 11B is a sectional view of the device lock of FIG. 11A taken from line B-B.

FIG. 11C is an end view of the device lock of FIG. 11A.

FIG. 12A is a perspective view another embodiment of a device lock.

FIG. 12B is an end view of the device lock of FIG. 12A.

FIG. 13 is a perspective view of one embodiment of an expander.

FIG. 14 is a flat view of one embodiment of a configuration that may be employed in fabrication of the expander of FIG. 13.

FIGS. 15A-C show side views of the expander of FIG. 13 in operation with an introducer sheath. FIG. 15A shows the expander in a non-expanded configuration and constrained inside an introducer sheath. FIG. 15B shows the expander in a partially expanded configuration and FIG. 15C shows the expander in a fully expanded configuration.

FIGS. 16A-D show the expander of FIG. 13 in operation with an introducer sheath and with a device lock. FIG. 16A shows the expander being loaded into an introducer sheath using a loading tool. FIG. 16B shows the expander and the loading tool. FIGS. 16C-D respectively show the expander in partially expanded and fully expanded configurations.

FIG. 17 illustrates a top view of one embodiment of an access kit.

### DETAILED DESCRIPTION

Generally, an introducer sheath can provide a minimally invasive access into a certain location inside the body of a patient. The introducer sheath includes a variable stiffness along its longitudinal profile. Various implementations may be employed to achieve the variable stiffness desired. The introducer sheath can be easily inserted and moved at various lengths, angles and/or directions inside the body, while avoiding or at least reducing trauma to the patient. The introducer sheath can be secured once it is positioned in the desired location and can expand a working space inside the body of a patient.

As one application, an introducer sheath can introduce devices and/or medical treatment products used in minimally invasive medical procedures. In one particular example, the introducer sheath can be useful for introducing such devices that employ a sub-xiphoid access in procedures performed on the heart. Such devices used in procedures performed on the heart can further include, for instance, a left atrial appendage closure device or a device used in a by-pass procedure.

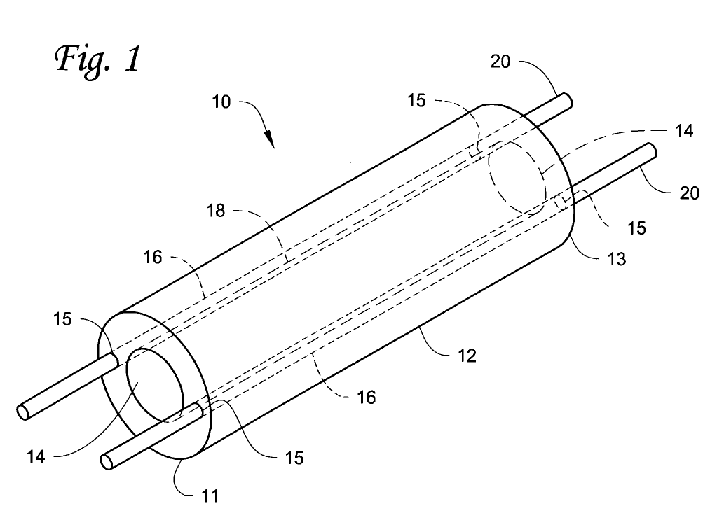

FIG. 1 illustrates a partial perspective view of one embodiment of an introducer sheath 10. The introducer sheath 10 provides a variable stiffness along the length of an elongated body 12. In this embodiment, the elongated body 12 may be generally flexible and includes opposite ends 11, 13 that are longitudinally disposed.

An opening 14 is disposed at each of the ends 11, 13, such that a channel 18 is defined longitudinally through the elongated body 12. In one embodiment, the channel 18 is configured to allow introduction of devices and/or other medical treatment products through the introducer sheath 10 to the inside of the body of a patient. The specific configuration and size of the channel 18 is not limited, so long as desired devices and/or other medical treatment products can be suitably introduced through the introducer sheath 10. As one example, an inside diameter of the introducer sheath is approximately 30 French. (0.390″ I.D.×0.020″ wall).

In one embodiment, the introducer sheath 10 may be produced from one or more polymer materials using an extrusion process. The polymer(s) used may have a constant durometer or may have different durometers. Some examples of polymers that may be used include Pebax®, polyamide, polyethylene, polyvinylchloride (PVC), and polypropylene. It will be appreciated that these polymers are merely exemplary, as a number of materials may be used to produce the introducer sheath 10, which may or may not include a polymer material and which may or may not involve an extrusion process, so long as the introducer sheath 10 is provided with variable stiffness and is suitable for use inside the body of a patient.

Lumens 16 are disposed in the wall of the elongated body 12. The lumens 16 provide spaces to insert support members 20 therethrough. In one embodiment, the lumens resemble channels longitudinally extending through the elongated body 12, and that are disposed within the wall structure of the elongated body 12 and outside the periphery of the channel 18. In one embodiment, the lumens 16 are extruded longitudinally through the wall of the elongated body 12 and include holes 15 at the ends 11, 13.

The lumens 16 are configured so that support members 20 may be inserted therethrough. When inserted, the support members 20 allow the introducer sheath 10 to vary its stiffness along the longitudinal profile (or length) of the elongated body 12. For example, the introducer sheath 10 can have a decreasing stiffness from the proximate end to the distal end. The stiffness of the support members 20 can be varied by using multiple support members of discrete lengths and differing stiffness. The stiffness of the support members 20 also can be varied by using a one length support member that has been treated, such as by a heat treatment to induce a varying stiffness along its length, or by grinding the outside diameter of the support member to remove material.

In assembly, the support members 20 are fed into the lumens 16 to induce variable stiffness along the elongated body 12. In yet another example, the support members 20 may be continuously extruded into the lumens 16 within the wall of the elongated body 12, such as during extrusion of the introducer sheath 10. Such extruded support members would also include a variable stiffness along the length of the lumens and therefore the elongated body. It will be appreciated that the specific manner in which the support members 20 are put inside the lumens 16 is non-limiting and that other methods may be employed that are equally suitable.

In FIG. 1, the support members 20 resemble rod-like structures. In some examples, the support members 20 may be metal or plastic rods or a combination of both. As shown, the elongated body 12 includes two lumens 16. Likewise, FIG. 1 shows two support members 20 inserted through the two lumens 16. It will be appreciated that the introducer sheath 10 is not limited to the specific structure shown and that one lumen or more than two lumens may also be employed if desirable and/or necessary. Likewise, it further will be appreciated that the number of support members 20 may vary as desirable and/or necessary, so long as there is a sufficient number of support members disposed in the number of lumens employed to achieve the varying stiffness desired.

As shown, the support members 20 are longer than the elongated body 12, however, this is merely for illustrative purposes to show one embodiment where the support members 20 are inserted through the length of the elongated body 12. It will be appreciated that the length of the elongated body 12 may be at least as long as, or longer, relative to each length of support member(s) used in each lumen 16.

In yet another example, an introducer sheath 600 may include a single lumen 616, which may be disposed within the wall structure of the elongated body 612 and just beyond the outside periphery of the channel 618 (e.g. FIG. 8). That is, the lumen 616 may be configured as a continuous void between the wall defining the channel 618 and the outer wall of the elongated body 612. In this configuration, a support member 620 may be configured as a cylindrical tube that can be inserted or formed within the wall structure of the elongated body 612, but outside of the channel 618.

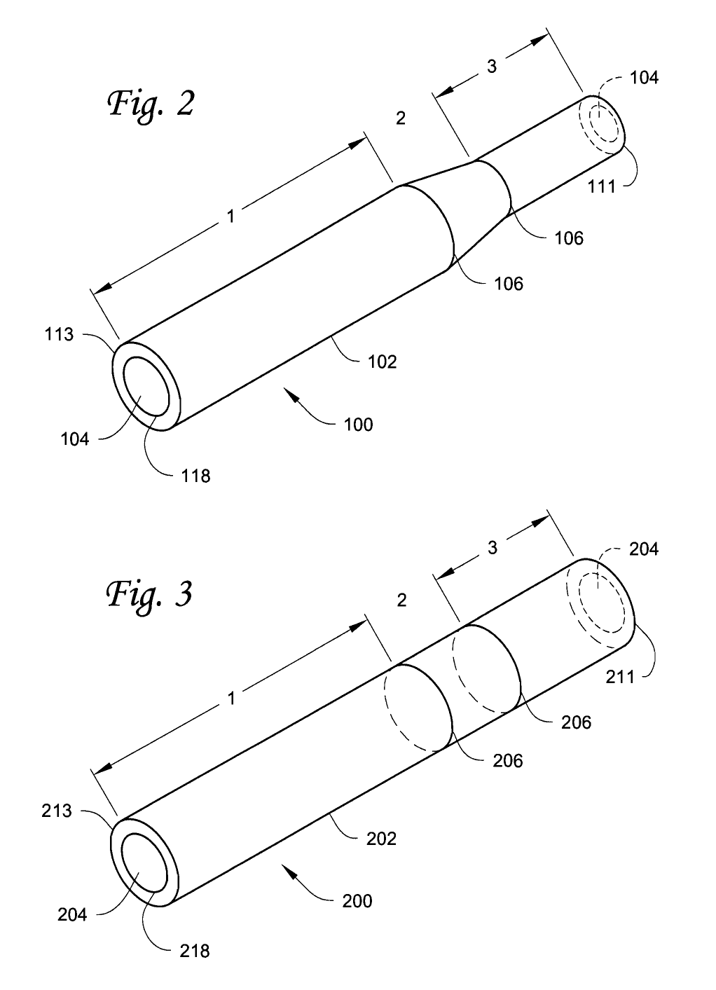

FIG. 2 illustrates a side view of another embodiment of an introducer sheath 100. The introducer sheath 100 includes an elongated body 102. As with elongated body 12, the elongated body 102 may be produced using an extrusion technique. The elongated body 102 defines openings 104 at ends 111, 113. As with introducer sheath 10, the introducer sheath 100 includes a channel 118 (not completely shown) longitudinally extending through the elongated body 102, similar to channel 18.

The introducer sheath 100 includes zones 1-3 that have different durometers. In one embodiment, the zones 1-3 have a decreasing durometer from end 113 to end 111. In this configuration, the flexibility of the elongated body 102 increases from end 113 to end 111, or for that matter the elongated body 102 becomes less stiff from end 113 to end 111. The introducer sheath 100 may also include transition zones 106 to allow for gradual change between the zones 1-3 if desired. In one embodiment, the transition zones 106 are configured as a tapered surface on the outer surface of the elongated body 102, where the end 111 has a thinner profile than end 113. The transition zones 106 can allow for a smooth change of stiffness along the elongated body 102, and may also avoid sharp edge surfaces in the overall introducer sheath profile and surface.

In one embodiment, the end 113 would be a proximate end and the end 111 would be a distal end. The proximate end would be operable for pushing the distal end into the body of a patient, where the proximate end precedes or trails the distal or lead end. In this configuration, the decreasing durometer of the introducer sheath 100 from end 113 to end 111 provides a more stiff (or less flexible) zone 1, while allowing easier pushing of the introducer sheath into the body. Zones 2 and 3 gradually have more flexibility (or less stiff) for easier articulation of the introducer sheath about bodily tissue, when the introducer sheath is inside the body of a patient. In one embodiment, zone 3 is most flexible (or least stiff).

As mentioned, variable stiffness can be accomplished through continuous extrusion, where the zones 1-3 of the introducer sheath 100 are changed on the extruder during the extrusion process. As a result, a variable stiffness can be achieved along the length of the elongated body 102.

As mentioned, the introducer sheath 100 can also provide for at least one transition zone 106. The transition zones 106 provide for a gradual change in stiffness, for example, as a result of the zones 1-3 having different durometers. It will be appreciated that the gradual change in stiffness can be tailored to introducer sheaths having various lengths.

It further will be appreciated that the length of any of the introducer sheaths described herein can be modified as necessary, so long as the introducer sheath has a sufficient length to allow introduction into a desired location of the body of a patient. It is one desire that an introducer sheath can introduce a desired device and/or medical treatment product(s) into the body of a patient. It may be another desire that the introducer sheaths described herein should not limit the function of the device and/or treatment product(s) meant to be introduced and used in the body.

FIG. 2 shows that the introducer sheath 100 includes an elongated body 102 with a decreasing thickness from end 113 to end 111 along its longitudinal profile. That is, FIG. 2 further shows the introducer sheath 100 with a reduced diameter at end 111 than at end 113. In this configuration, the change in thickness dimension can also help vary the stiffness (or flexibility) along the longitudinal profile of the elongated body 102.

As with introducer sheath 10, the introducer sheath 100 may be produced using one or more polymers along its length. The polymer(s) used may have a constant durometer or may have different durometers. Some examples of polymers that may be used include Pebax, polyamide, polyethylene, polyvinylchloride (PVC), and polypropylene. It will be appreciated that these polymers are merely exemplary, as a number of materials may be used to produce the introducer sheath 100, which may or may not include a polymer material and which may or may not involve an extrusion process, so long as the introducer sheath 100 is provided with variable stiffness and is suitable for use inside the body of a patient.

FIG. 3 illustrates a side view of another embodiment of an introducer sheath 200. The introducer sheath 200 includes an elongated body 202 having ends 211, 213. The elongated body 202 defines openings 204 at ends 211, 213. As with the previous introducer sheaths, the introducer sheath 200 also includes a channel 218 (not completely shown) longitudinally extending through the elongated body 202. Differently from the introducer sheath 100, the elongated body 202 of introducer sheath 200 substantially does not change its diameter or thickness along the longitudinal profile. Rather, the elongated body 202 substantially is maintained with the same overall profile diameter or thickness from end 213 to end 211.

The introducer sheaths 10, 100, 200 described are shown substantially cylindrical shaped. It will be appreciated that the introducer sheaths are not limited to this specific shape, so long as the introducer sheaths described can be safely inserted into the body of the patient and used therein.

Turning back to FIG. 3, introducer sheath 200 has a variable stiffness along the elongated body 202. Zones 1-3 are provided, such that in one embodiment zone 1 is the most stiff while zones 2 and 3 are increasingly less stiff (i.e. have decreasing stiffness or more flexibility).

In one embodiment, the end 213 would be a proximate end and the end 211 would be a distal end. The proximate end would be operable for pushing the distal end into the body of a patient, where the proximate end precedes or trails the distal or lead end. In this configuration, the decreasing durometer of the introducer sheath 200 from end 213 to end 211 provides a stiffer (or less flexible) zone 1, while allowing easier pushing and insertion of the introducer sheath 200. Zones 2 and 3 gradually have more flexibility (or less stiffness) for allowing easier articulation about bodily tissue, when the introducer sheath 200 is inside the body of a patient. As with introducer sheath 100, zone 3 may be the most flexible (or least stiff) in one embodiment.

In one embodiment, zone 1 may be constructed of a stiff polymer (Pebax a resin, polyamide, polyethylene, polypropylene) having a relatively higher stiffness than zones 2 and 3. Zone 2 may be constructed of a lower durometer resin that has a relatively lower stiffness than zone 1. Zone 3 may be constructed of yet a lower durometer resin that has a relatively lower stiffness (higher flexibility) than zones 1 and 2. As one example only, zone 1 may be constructed with stiff material such as a 72D durometer resin, and zone 2 may be constructed of a lower durometer material such as a 55D durometer resin, while zone 3 may be constructed of yet a lower durometer material such as a 35D durometer resin. It will be appreciated that the specific material used, as well as, the specific durometers employed may be modified as suitable and/or necessary.

It further will be appreciated that any portion of the elongated body having a particular durometer zone may be configured as long as desired and as suitable for the procedure the introducer sheath is to be used. In one embodiment, the front ½ to ⅔ portion (toward the distal end) of the introducer sheath may be made of a more flexible material that can allow articulation of the sheath over tissues inside the body. The back portion (toward the proximate end) may be made of a more stiff or less flexible material to facilitate pushing of the introducer sheath inside a patient and for securing the introducer sheath to a patient, such as on his/her skin. It is one desire that the introducer sheaths described herein allow for introduction of a device and/or medical treatment product(s), when the introducer sheath is secured in position.

As mentioned, variable stiffness can be accomplished through continuous extrusion. In the embodiment of FIG. 3, the durometer or stiffness of the material used for zones 1-3 of the introducer sheath 200 is changed on the extruder during the extrusion process to achieve variable stiffness. The introducer sheath 200 can also include at least one transition zone 206. The transition zones 206 provide for a change in stiffness of the elongated body 202, as a result of the zones 1-3 having different stiffness. As with introducer sheath 100, it will be appreciated that the change in stiffness can be constructed to accommodate introducer sheaths having various lengths, and that the length of the introducer sheath can be modified as necessary. Likewise, the specific length of the introducer sheath 200 is not limited, so long as the introducer sheath 200 has a sufficient length to allow introduction into a desired location of the body of a patient. It may be desired that the introducer sheath 200 has a length so as to be able to introduce a desired device and/or medical treatment products into the body of a patient. It may also be desired that the introducer sheath 200 should not limit the function of the device and/or treatment product(s) meant to be introduced.

As with the other introducer sheaths described herein, the introducer sheath 200 may be produced by a variety of polymers along its length. The polymers used have different durometers to achieve the varying stiffness along the elongated body 202. It will be appreciated that a number of materials such as already described may be used to produce the introducer sheath 200, and which may or may not include a polymer material and which may or may not involve an extrusion process, so long as the introducer sheath 200 is provided with variable stiffness and is suitable for use inside the body of a patient.

In another embodiment, any of the introducer sheaths described herein may be made to be radio-opaque, such as by the addition of a radiopaque material into the polymer or material used to construct the introducer sheath. Employing a radiopaque material can allow the use of fluoroscopy, such as during a clinical procedure to place the sheath inside the patient's body. In one embodiment, a radiopaque material(s) is compounded into the material used to produce the introducer sheath. As one example a radiopaque material is compounded into each section of material of the introducer sheath. As another example, the radiopaque material is dispersed evenly throughout the length of the introducer sheath, where a percentage of radiopaque material can be loaded into the compounded material used to produce the introducer sheath.

As mentioned, the introducer sheaths described may be configured with a proximate end and a distal end, where the proximate end includes the most stiff (or less flexible) zone and the distal end includes the most flexible (or less stiff) zone. In such a configuration, the variable stiffness decreases from the proximate end to the distal end. That is, the proximate end (i.e. zone 1) is more stiff than the distal end, where the introducer sheath is increasingly more flexible (i.e. zones 2 and 3) toward the distal end. In FIGS. 2 and 3, three zones 1-3 are shown. It will be appreciated that the number of zones is merely exemplary as there may be two zones or more than three zones if desired or necessary.

In another embodiment, the introducer sheath may be constructed with a braided reinforcement. That is, to ensure that the inside diameter remains open, the introducer sheath may be constructed with a braid (not shown) to support the introducer sheath's elongated body and to prevent the introducer sheath from collapsing and reducing its inside diameter. Such braid reinforcement structures are well known and are available for the production of various tubing and would also be available for producing an elongated body of an introducer sheath with added reinforcement. In one embodiment, the braid reinforcement may be disposed on an inner surface of the introducer sheath's body, or disposed as a laminate between an inner and outer material of the introducer sheath, or embedded within the material of the elongated body. It will be appreciated that the braid reinforcement may be disposed along the length of the introducer sheath, or on portions thereof.

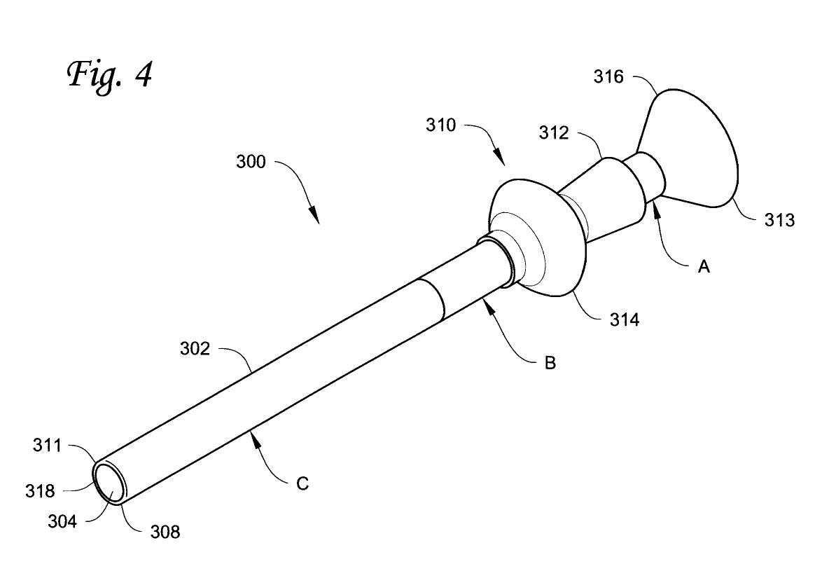

Turning to FIG. 4, another embodiment of an introducer sheath 300 is shown having a retention mechanism 310 and a device lock 316. The retention mechanism 310 can stabilize or secure the introducer sheath 300 in place, once the introducer sheath 300 has been positioned in the desired location for use. The device lock 316 can stably hold desired device(s) and/or medical treatment product(s), once introduced through the introducer sheath 300 and put in position.

As with the other introducer sheaths described, the introducer sheath 300 includes an elongated body 302 with ends 311, 313. In one embodiment, the end 313 is a proximate end and the end 311 is a distal end. The distal end 311 includes an opening 304, where a channel 318 (not completely shown) longitudinally extends through the elongated body 302. It will be appreciated that the introducer sheath 300 also includes an opening (not shown in FIG. 4) at the proximate end 313 similar to the other introducer sheaths described. Likewise, the elongated body 302 includes zones A-C, which are configured with differing durometers to vary the stiffness of the introducer sheath 300. In one embodiment, the zones A-C include relative stiffness with respect to each other, for example where the elongated body 302 includes a varying stiffness that is decreasingly stiff from the proximate end 313 to the distal end 311.

In one embodiment, the retention mechanism 310 includes a stop component 312 connected to the elongated body 302 that prevents inward movement of the introducer sheath 300, such as after a portion of the introducer sheath 300 has been inserted and positioned in the body of a patient. The retention mechanism 310 also includes a stop component 314 connected to the elongated body 302 that prevents outward movement, such as after a portion of the introducer sheath 300 has been inserted and positioned in the body of a patient.

In one embodiment, the stop component 312 is disposed about the elongated body 302 distal of the proximate end 313. The stop component 312 has an increased diameter relative to the elongated body 302, so as to limit the amount the introducer sheath can be inserted. That is, the stop component 312 substantially resides outside the body of a patient as insertion of the introducer sheath 300 is limited by the increased diameter structure of the stop component 312. In one embodiment, the stop component 312 includes an increasing diameter toward the proximate end 313, where that the stop component 312 has a tapered configuration.

Likewise, the stop component 314 is configured such that it may have an increased diameter when the introducer sheath 300 has been inserted and positioned in the body of a patient. As shown, the stop component 314 is an expandable member disposed about the outer surface of the elongated body 302. In one embodiment, the stop component 314 is an inflatable balloon, which can be inflated once the introducer sheath 300 has been inserted and positioned at a desired location in the body. In this configuration, the introducer sheath 300 can be prevented from moving outward and inward and from being dislodged once it has been put in place. That is, the stop component 314 would reside within the body of a patient to prevent unwanted release of the introducer sheath 300.

In yet another embodiment, the stop components 312, 314 are adjustable along the elongated body 302. For example, the stop components 312, 314 can be slidably and axially moved along the length of the elongated body 302, so as to adjust the length or amount the introducer sheath 300 can be inserted. In this configuration, the retention mechanism is sutureless, where the amount of insertion of the introducer sheath 300 can be adjusted based on the positioning of the stop components 312, 314. In operation, for example, the stop component 312 can be secured or locked in position so as to stay in place, and the introducer sheath 300 can be inserted to a certain length allowed by the stop component 312. Once the introducer sheath 300 is inserted to the extent allowed by the stop component 312, the stop component 314 can be adjusted and secured in position so as to stay in place. The stop component 314 can then be inflated to prevent release of the introducer sheath 300. As stop component 314 is inflated, the inside diameter is reduced and forms a tight fit to the outside of sheath 300.

It will be appreciated that retention mechanism 310, including the stop components 312, 314, is not limited to the specific structure and configuration shown in FIG. 4, so long as the retention mechanism 310 can stabilize the introducer sheath 300 in place after a desired portion has been inserted and positioned in a desired location within the body of a patient.

The device lock 316 is disposed toward the proximate end 313. In one embodiment, the device lock 316 is a hub-like structure disposed at the proximate end 313, which can be rotated to secure a device and/or medical treatment product(s) that are desired for introduction through the introducer sheath 300.

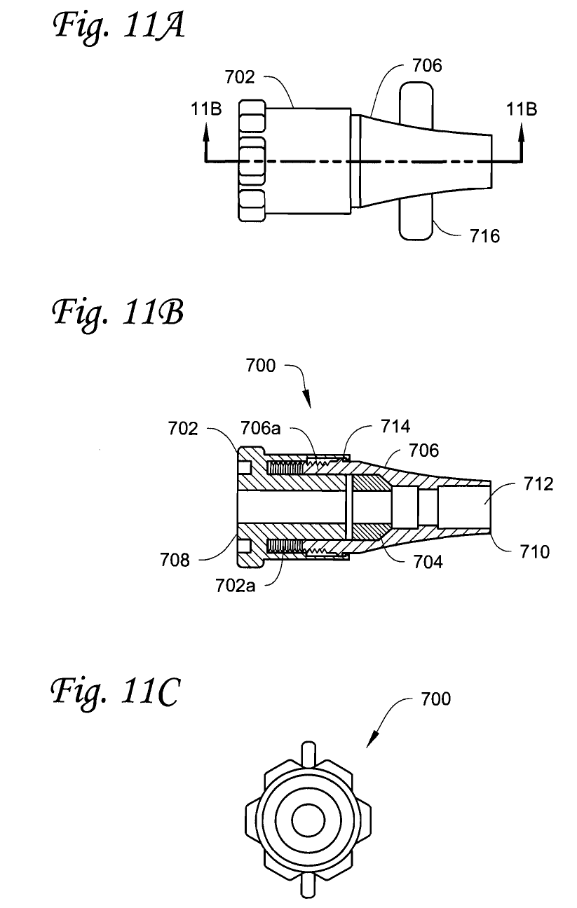

In one embodiment, as you rotate the device lock 316 there may be a polymer donut (not shown) inside the device lock 316 that is compressed. This compression results in the inside diameter of the polymer donut being reduced, which then tightens the polymer donut around the shaft of the device or medical treatment product being inserted through the introducer sheath. It will be appreciated that reduction of the inside diameter of the polymer donut may be accomplished any number of ways by any suitable motion or mechanism, so long as such technique employed results in a reduction of the inside diameter of the polymer donut. An example of such a donut construction is illustrated and described in more detail with respect to FIGS. 11A-C below. As mentioned, the device lock 316 can stably hold a device and/or medical treatment product(s) for use inside the body, after being introduced through the introducer sheath and put in position.

It will be appreciated that the device lock 316 may be constructed a number of ways, other than with a polymer donut construction. As some other examples, a tight fitting diaphragm or a cam structure with pinch or squeeze members may be employed as lock structures to be activated by a device lock.

In FIG. 4, the distal end 311 is configured as a distal tip 308 to allow for ease of navigation, so that the introducer sheath 300 can smoothly pass over tissue surfaces inside the body of the patient. It is one desire that the distal tip 308 can prevent damage to tissue inside the body when the introducer sheath is being positioned therein. It is another desire that the distal tip 308 can also help reduce patient trauma.

In one embodiment, the distal tip 308 can be a cushion-like structure or “bumper” to provide non-traumatic cushion when the introducer sheath 300 is navigated about tissue inside a patient, for example when passing over the surface of the heart. In one embodiment, the distal tip is a soft flexible material. It will be appreciated that the distal tip 308 may be the material of the last or most distal zone of the elongated body or may be constructed of a separate material. In one embodiment, the distal tip 308 may be configured of about 2 to 6 mm in length from the distal end 311 toward the proximate end 313. In another embodiment, the distal tip 308 is radiused or rounded to remove or at least minimize any sharp edge surfaces at the distal end 311. (See for example FIG. 7.)

FIG. 7 illustrates a partial side sectional view of one embodiment of a distal end 5 which may be employed in any of the introducer sheaths described herein. As with the other introducer sheaths described, introducer sheath 7 includes an opening 4 with a channel 6 longitudinally extending in the elongated body 2. FIG. 7 further shows a distal tip 8 having a rounded configuration. As one example, the distal tip 8 may also include a lubricious coating thereon. Some non-limiting examples of a coating include a silicon and/or hydrophilic material.

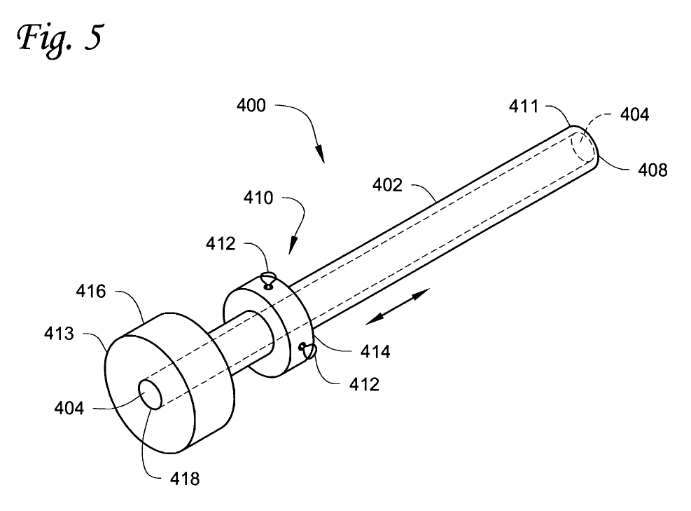

FIG. 5 illustrates a side view of another embodiment of an introducer sheath 400. The introducer sheath 400 shows another embodiment of a retention mechanism 410 and a device lock 416. As with the retention mechanism 310, the retention mechanism 410 can stabilize or secure the introducer sheath 400 in place, once the introducer sheath 400 has been positioned in the desired location for use. The device lock 416 can stably hold a device and/or medical treatment product(s) to be used inside the body, once introduced through the introducer sheath 400 and put in position.

As with the other introducer sheaths described, the introducer sheath 400 includes an elongated body 402 with ends 411, 413. In one embodiment, the end 413 is a proximate end and the end 411 is a distal end. The ends 411, 413 include openings 404, where a channel 418 (not completely shown) longitudinally extends through the elongated body 402. It will be appreciated that the introducer sheath 400 may also include zones (not indicated) as the other introducer sheaths, which are configured with differing durometers to vary the stiffness of the introducer sheath 400. In one embodiment, the zones may include a relative stiffness with respect to each other, for example where the elongated body 402 includes a varying stiffness that is decreasingly stiff from the proximate end 413 to the distal end 411.

In one embodiment, the retention mechanism includes using sutures to secure the introducer sheath 400 in place. The retention mechanism 410 prevents inward and outward movement of the introducer sheath 400, such as after a portion of the introducer sheath 400 has been inserted and positioned in the body of a patient.

In one embodiment, the retention mechanism 410 includes a stabilizer member 414 that is longitudinally adjustable along the length of the elongated body 402. As with the stop components described above, the stabilizer member 414 can be slidably and axially moved along the length of the elongated body 402. Such a configuration allows for adjusting the length or amount the introducer sheath 400 can be inserted into a patient's body. In one embodiment, the stabilizer member 414 is a structure disposed about the outer surface of the elongated body 402, so as to give the introducer sheath 400 a larger diameter wherever the stabilizer member 414 is positioned. In this configuration, the stabilizer member 414 also acts to provide a stop as to how far the introducer sheath 400 can be inserted into the body. As shown, the stabilizer member 414 resembles a donut-like structure. As with the previously described stop components, the stabilizer member 414 can be secured or locked into place on the elongated body 402 once it is placed in a desired location. It will be appreciated that the stabilizer member 414 is not limited to the specific structure shown, so long as the stabilizer member can be moved to adjust the amount that the introducer sheath 400 can be inserted and can be secured to a patient to stabilize the introducer 400.

The retention mechanism 410 includes connecting members 412 disposed on the stabilizer member 414. The connecting members 412 are operable for securing the stabilizer member to the body of a patient once the introducer sheath 400 has been put in place. As one example, the connecting members 412 resemble loop like structures on the stabilizer member 414, such that well known and commercially available sutures can be applied through the connecting members 412 to secure the stabilizer member 414 to the body of a patient. In FIG. 5, three connecting members 412 are illustrated. It will be appreciated that the number of connecting members shown is merely exemplary and that more or less than three connecting members 412 may be suitably employed as desirable or necessary.

In operation for example, the introducer sheath 400 can be inserted to a certain length as allowed by the stabilizer member 414. The stabilizer member 414 can be locked in place on the elongated body 402. In one embodiment, stabilizing member 414 is locked to introducer sheath 400 through a polymer donut that reduces its inside diameter. In one embodiment, the inside diameter of the polymer donut may be reduced as the result of compression of the polymer donut. Once the introducer sheath 400 is inserted to the extent allowed by the stabilizer member 414 and put in a desired position, the stabilizer member 414 can be secured in position by suturing down the stabilizer member 414 using a suture material and the connecting members 412.

As a result of such a configuration, the introducer sheath 400 effectively includes an adjustable suture tie down to secure the introducer sheath to a patient. As shown, the adjustable tie down includes a sliding element (i.e. stabilizer member 414) that can be adjusted to allow a certain length of the introducer sheath to be introduced into a patient. The sliding element includes tie offs (i.e. connecting members 412) to secure the introducer sheath to the patient, such that the introducer sheath does not undesirably get dislodged or moved out of place.

It will be appreciated that retention mechanism 410, including the stabilizer member 414 and securing members 412, is not limited to the specific structure and configuration shown in FIG. 5, so long as the retention mechanism 410 can stabilize the introducer sheath 400 in place after a desired portion has been positioned to a desired location in the body of a patient. It is one desire that any of the retention mechanisms described can be disabled so that it can be easily removed, while minimizing or preventing trauma to a patient.

Similar to the device lock 316 of introducer sheath 300, the device lock 416 is disposed toward the proximate end 413. In one embodiment, the device lock 416 is a hub-like structure disposed at the proximate end 413, which can be rotated to secure a device(s) and/or medical treatment product(s) that are desired for introduction through the introducer sheath 400. In one embodiment, as the device lock 416 is rotated, there may be a polymer donut inside the device lock 416 that is compressed. This compression can result in the inside diameter of the polymer donut being reduced, which then tightens the polymer donut around the shaft of the device or medical treatment product being used. See FIGS. 11A-C further described herein. It will be appreciated that reduction of the inside diameter of the polymer donut may be accomplished any number of ways by any suitable motion or mechanism, so long as such technique employed results in a reduction of inside diameter of the polymer donut. As mentioned, the device lock 416 can stably hold device(s) and/or medical treatment product(s), once introduced through the introducer sheath 400 and put in position.

As with introducer sheath 300, the introducer sheath 400 includes a distal end 411 having a distal tip 408 to allow for ease of navigation, so that the introducer sheath 400 can smoothly pass over tissue surfaces inside the body of the patient. It is one desire that the distal tip 408 can prevent damage to tissue inside the body when the introducer sheath 400 is being positioned therein. It is another desire that the distal tip 408 can also help reduce patient trauma.

As with the distal tip 308, the distal tip 408 can be a cushion-like structure or “bumper” to provide non-traumatic cushion when the introducer sheath 400 is navigated inside a patient, for example when passing over the surface of the heart. In one embodiment, the distal tip is a soft flexible material. It will be appreciated that the distal tip 408 may be the material of the last or most distal zone of the elongated body or may be constructed of a separate material. In yet another embodiment, the distal tip 408 may be configured of about 2 to 6 mm in length from the distal end 411 toward the proximate end 413. In another embodiment, the distal tip 408 is radiused or rounded to remove or at least minimize sharp edge surfaces at the distal end 411. (See e.g. FIG. 7.) In yet another embodiment, the distal tip 408 may also include a lubricious coating thereon. Some non-limiting examples of a coating include a silicon and/or hydrophilic material.

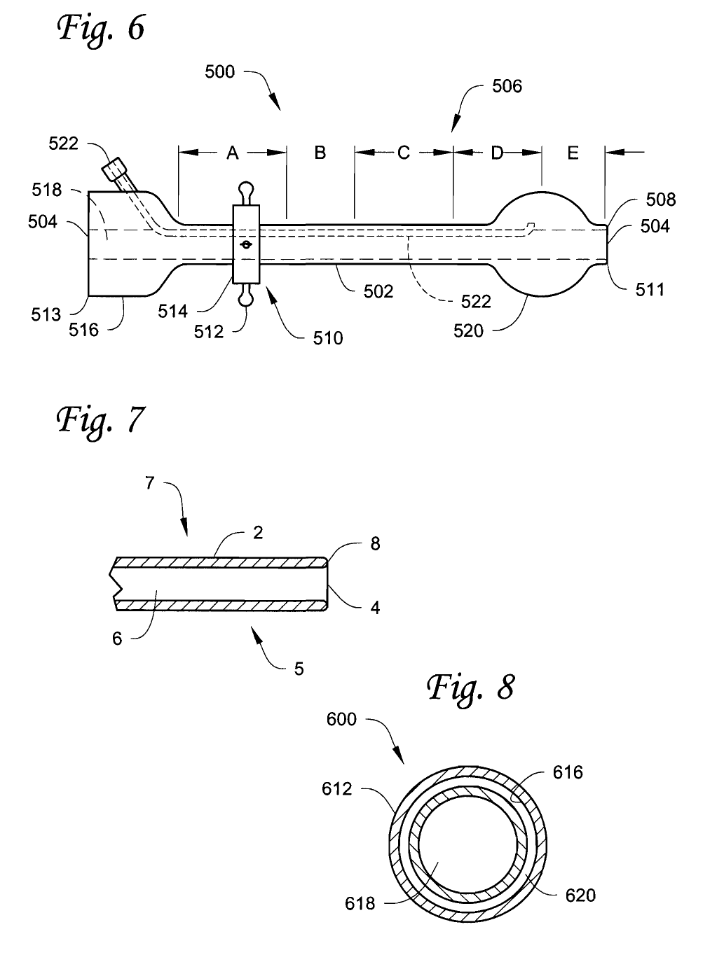

FIG. 6 illustrates a side view of another embodiment of an introducer sheath 500. In one embodiment, the introducer sheath 500 includes an expander 520. The expander 520 is operable for expanding a space inside the body of a patient at a targeted anatomy, so as to provide a working space to allow easier performance of a procedure inside the body (further discussed below).

As with the other introducer sheaths described, the introducer sheath 500 includes an elongated body 502 with ends 511, 513. In one embodiment, the end 513 is a proximate end and the end 511 is a distal end. The ends 511, 513 include openings 504, where a channel 518 (not completely shown) longitudinally extends through the elongated body 502. The introducer sheath 500 also includes zones 506 shown as A-E. As with the other introducer sheaths, the zones 506 are configured with differing durometers to vary the stiffness of the introducer sheath 500 along its longitudinal profile.

In one embodiment, the different zones 506 shown by A-E may include a relative stiffness with respect to each other, for example where the elongated body 502 includes a varying stiffness that is decreasingly stiff from the proximate end 513 to the distal end 511.

The introducer sheath includes a retention mechanism 510. In one embodiment, the retention mechanism 510 is similar to the retention mechanism 410, using sutures to secure the introducer sheath 500 in place. The retention mechanism 510 prevents inward and outward movement of the introducer sheath 500, such as after a portion of the introducer sheath 500 has been inserted and positioned in the body of a patient.

The retention mechanism 510 includes a stabilizer member 514 longitudinally adjustable along the length of the elongated body 502. For example, the stabilizer member 514 can be slidably and axially moved along the length of the elongated body 502, so as to adjust the length or amount the introducer sheath 500 can be inserted, and can be secured or locked on the elongated body 502 once it is placed in a desired position. In one embodiment, the stabilizer member 514 is a “donut-like” structure disposed about the outer surface of the elongated body 502. The stabilizer member 514 gives the introducer sheath 500 a larger diameter wherever the stabilizer member 514 is positioned. In this configuration, the introducer sheath 500 can be adjusted based on the positioning of the stabilizer member 514. The stabilizer member 514 also may act to provide a stop as to how far the introducer sheath can be inserted into the body. It will be appreciated that the stabilizer member 514 is not limited to the specific structure shown, so long as the stabilizer member is movable to adjust the amount that the introducer sheath can be inserted.

The retention mechanism 510 also includes connecting members 512 disposed on the stabilizer member 514. The connecting members 512 are operable for securing the stabilizer member 514 to the body of a patient once the introducer sheath 500 has been put in place. In one embodiment, the connecting members 512 resemble loop like structures on the stabilizer member 514, such that sutures can be applied through the connecting members 512 to secure the stabilizer member 514 to the body of a patient. In FIG. 6, three connecting members 512 are illustrated. It will be appreciated that the number of connecting members shown is merely exemplary and that more or less than three connecting members 512 may be suitably employed as desirable or necessary.

In operation, the retention mechanism 510 operates similarly as the retention mechanism 410. The stabilizer member 514 can be locked in place on the elongated body 502. As in other stabilizing members described, stabilizing member 514 may be locked to the introducer sheath 500 by reducing an inside diameter of a polymer donut. The polymer donut's inside diameter is reduced as the result of compression. Once the introducer sheath 500 is inserted to the extent allowed by the stabilizer member 514 and put in a desired position, the stabilizer member 514 can be secured in position by suturing down the stabilizer member 514 to a patient using a suture material and the securing members 512.

As a result of such a configuration, the introducer sheath 500 also provides an adjustable suture tie down to secure the introducer sheath to a patient. As shown, the adjustable suture tie includes a sliding element (i.e. stabilizer member 514) that can be adjusted to allow a certain length of the introducer sheath to be introduced into a patient. The sliding element includes tie offs (i.e. connecting members 512) to secure the introducer sheath to the patient, such that the introducer sheath does not undesirably get dislodged or moved out of place.

It will be appreciated that retention mechanism 510, including the stabilizer member 514 and securing members 512, is not limited to the specific structure and configuration shown in FIG. 6, so long as the retention mechanism 510 can stabilize the introducer sheath 500 in place after a portion of the introducer sheath has been positioned to a desired location in the body of a patient. It is one desire that any of the retention mechanisms described can be disabled for easy removal, while minimizing or preventing trauma to a patient.

As with the other device locks disclosed, the device lock 516 is disposed toward the proximate end 513. In one embodiment, the device lock 516 is a hub-like structure disposed at the proximate end 513, which can be rotated to secure a device and/or medical treatment product(s) that are desired for introduction through the introducer sheath 500. In one embodiment of operation, as the device lock 516 is rotated there is a polymer donut inside the device lock 516 that can be compressed. This compression can result in the inside diameter of the polymer donut being reduced, which then tightens the polymer donut around the shaft of the device or medical treatment product being used. See FIGS. 11A-C and descriptions herein. It will be appreciated that any motion or mechanism which can will result in a reduction of an inside diameter of the polymer donut may be employed. As mentioned, the device lock 516 can stably hold a device and/or medical treatment product(s) to be used inside the body, once introduced through the introducer sheath 500 and put in position.

As with the other introducer sheaths described, the distal end 511 also includes a distal tip 508 to allow for ease of navigation, so that the introducer sheath 500 can smoothly pass over tissue surfaces inside the body of the patient. It is one desire that the distal tip 508 can prevent damage to tissue inside the body when the introducer sheath is being positioned therein. As another example, it is one desire that the distal tip 508 can also help reduce patient trauma that may be associated with insertion and navigation of the introducer sheath 500.

The distal tip 508 can be a cushion-like structure or “bumper” to provide non-traumatic cushion when the introducer sheath 500 is navigated inside a patient, for example when passing over the surface of the heart. In one embodiment, the distal tip is a soft flexible material. It will be appreciated that the distal tip 508 may be the material of the last or most distal zone of the elongated body or may be constructed of a separate material. In yet another embodiment, the distal tip 508 may be configured of about 2 to 6 mm in length from the distal end 511 toward the proximate end 513. In another embodiment, the distal tip 508 may also be radiused or rounded to remove or at least minimize sharp edge surfaces at the distal end 511. (See e.g. FIG. 7.) In yet another embodiment, the distal tip 508 may also include a lubricious coating thereon. Some non-limiting examples of a coating include a silicon and/or hydrophilic material.

Turning to the expander 520, the expander 520 in one embodiment extends out of the introducer sheath 500 proximate the distal end 511. As shown, the expander 520 is disposed proximate the distal end 511, and expands out of the outer surface of the elongated body 502. It is one desire that the expander 520 neither should be hindered by the introducer sheath 500, nor should it hinder use of any device and/or medical treatment products being introduced.

As some non-limiting examples, the expander 520 may be a balloon or a self-expanding structure. In the example of the expander 520 being a balloon, the balloon is expanded through a port 522 having an inflation lumen. As shown the port 522 extends as an inflation lumen running along the elongated body 502 and to the expander 520. Prior to expansion, the balloon is bonded to the introducer sheath 500 and lies against the outer surface of the introducer sheath 500. As some examples, the balloon is bonded using either an adhesive or thermal bonding. It will be appreciated that bonding the balloon to the outer surface of the elongated body can be accomplished a number of ways, and the technique used can vary depending on the final material selected of the elongated body and balloon. In yet another embodiment, the surface on the elongated body to which the balloon is bonded may range from about 2 to 6 mm from end to end.

It will further be appreciated that the expander can be symmetrically or asymmetrically deployed. That is, where the expander tends to be disposed on one side or portion of the sheath in its expanded configuration than on another side or portion of the sheath. If the expander is to be asymmetrically deployed, then the sheath can be identified with a mark to indicate how to orient the elongated body. It will be appreciated that orientation may not be a concern where the expander is symmetrical, because the expander is evenly disposed about the sheath when the expander is in its expanded configuration.

It will be appreciated that the expander 520 is not limited to the specific structure shown in FIG. 6, and that other configurations and arrangements may be employed that are equally or more suitable. The expander 520 is operable to open a space inside the body of a patient to create a working space that allows easier performance of a procedure inside the body. In one exemplary implementation, the expander 520 can be operable for opening the pericardial space of the heart of a patient to perform certain operations therein.

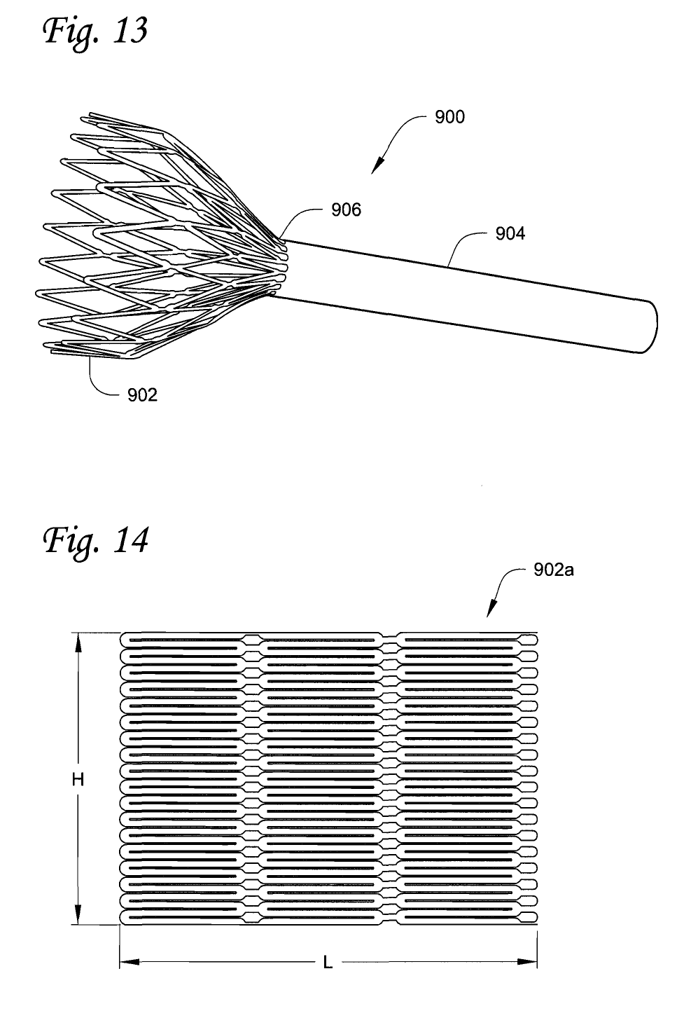

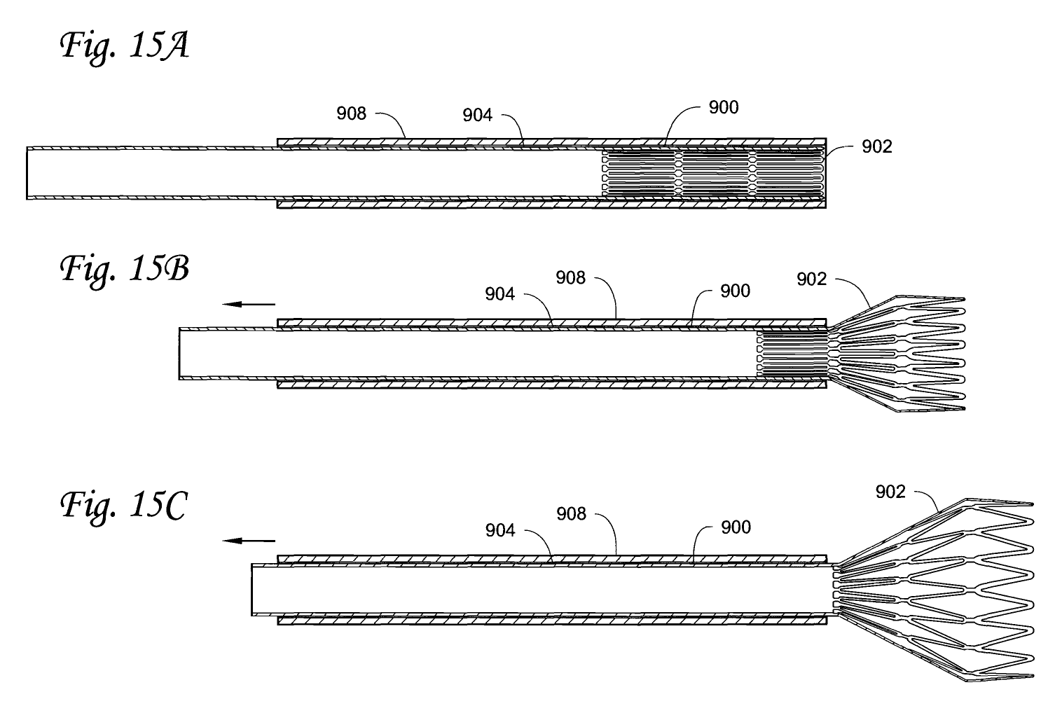

In other embodiments, the expander may be a self-expanding structure. One embodiment can be that the expander includes an expandable portion, such as a collapsible cage-like structure. The cage-like structure can be covered by a sheath that passes either over the outside diameter of any of the introducer sheaths described, or within the inside diameter of any of the introducer sheaths described. It will be appreciated that the introducer sheath itself can be used as the cover. For example, as the sheath is pulled back to uncover the expander, the cage-like structure will expand to a desired diameter. The cage can be reduced in diameter by recovering with the sheath. FIGS. 13 and 15A-15C herein further describe an expander 900 as a self-expanding structure.

As shown in FIG. 6, the introducer sheath 500 includes an elongated body 502 having variable stiffness from zones 506 (A-E), a retention mechanism 510, a device lock 516 mechanism, an expander 520, and a distal tip 508. The introducer sheath 500 provides one embodiment of an all in one sheath having all of the advantages described.

Turning back to the device lock, FIGS. 11A-C illustrate one embodiment of a device lock 700 and its structure, which may be employed with any of the introducer sheaths described herein and may replace any of the device lock structures previously described. FIGS. 11A-11C show the device lock 700 configured as an adapter structure for attaching to an elongated body of various sheaths and locking a medial device in a desired position. In one embodiment, the device lock 700 resembles a hub-like structure as described above. The device lock includes a proximal end 708 and a distal end 710. A cap 702 is disposed at the proximate end 708 and a body 706 resides at the distal end 710. The body 706 is adapted for attachment to an elongated body of an introducer sheath. As one example, the body includes a sheath attachment 712 that can be mated with an introducer sheath, where an elongated body can be inserted into the sheath attachment 712. It will be appreciated that the specific configuration of the body 706 and sheath attachment 712 is not limited and may be modified to be suitably attached to any of the elongated bodies described herein.

In one embodiment, the cap 702 and the body 706 are connected by a threaded engagement. Cap 702 includes an internal thread portion 702a that can threadedly connect with thread portion 706a. As shown in FIG. 11A, tab members 716 may be used to aid in tightening the cap 702, and is disposed at sides of the body 706. The tab members 716 can be useful for holding the body 706 in place while the cap 702 is being tightened. It will be appreciated that the configuration in which the cap 702 and body 706 are attached is merely exemplary as other configurations may be employed that are equally or more suitable.

The cap 702 and the body 706 contain a lock member 704. In one embodiment, the lock member 704 may be a donut-like structure as described above, where the lock member 704 resides inside the body 706. As shown, the lock member 704 resides between the cap 702 and the body 706 and in a longitudinal direction between proximate and distal ends 708, 710. When the device lock 700 is activated, the donut-like structure of the lock member 704 can be made to compress onto and about a medical device passing through device lock 700 and into the elongated body of the introducer sheath. The medical device can be secured in this manner, such as by an interference fit. In one embodiment, the device lock 700 may be activated, for example, by rotating the cap 702 with respect to the body 706, such that the relative rotation imparts a compressive effect on the lock member 704, to thereby compress and hold a desired medical device in place. It will be appreciated that the manner of attachment for the cap 702 and body 706 is not limited to the specific structure shown, as long as the device lock 700 can be effective for compressing the lock member 704 onto a medical device inserted through a sheath.

In yet another embodiment, the device lock 700 includes a snap feature 714. The snap feature 714 can help to ensure the cap does not fall off the body when the cap is not tight. The snap feature 714 resembles a bump-like structure or projection about the outer surface of the body 706. The snap feature 714 provides for the cap 702 to be pushed onto the body 706 in a snap fit configuration, where the body 706 is inserted past an annularly inward projection on the cap 702.

As described, the lock member 704 may be a donut-like structure that has an inner annular diameter to allow a medical device to be inserted therethrough, and that can be compressed onto such a device to lock it in place. As shown, the lock member 704 is inside body 706 and is compressed as the cap 702 receives the body 706. In one embodiment, the lock member 704 is a silicon rubber material or may be any compressible polymer material. The lock member 704 further includes an outer annular diameter having a distal taper portion that engages a taper portion inside the body 706. The cap 702 includes a shoulder that can push the lock member 704 toward the body, such that movement of the taper portion of the lock member 704 relative to the taper portion of the body 706, allows the lock member 704 to be compressed. That is, the lock member 704 can be compressed, as the cap 702 and body 706 are connected by the thread engagement. It will be appreciated that the lock member 704 may be constructed of a variety of materials, and is not limited to a silicon rubber. The lock member may be any material that can be suitably compressed onto a medical device to hold and secure it within an introducer sheath.

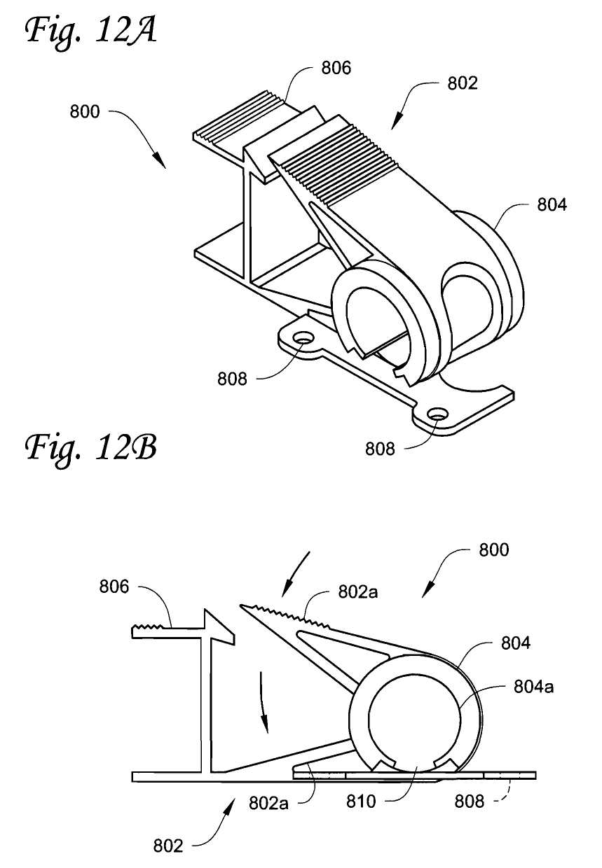

FIGS. 12A-B illustrate another embodiment of a device lock 800 for locking a device in place that is used in conjunction with an introducer sheath, and that also may be configured for use with an elongated body of an introducer sheath. That is, the device lock 800 may be useful for securing an introducer sheath, once the sheath is placed in its desired position. The lock device 800 includes a lock member 804 and a lock actuator 802. As shown, the lock member 804 may be a donut-like structure that is open or includes a void 808. As one example only, the lock member 804 is a silicon rubber material. It will be appreciated that the lock member 804 may be constructed of a variety of materials, and is not limited to a silicon rubber or a compressible polymer material. In general, the lock member 804 may be any material that can be suitably compressed onto the elongated body to hold and secure the introducer sheath or onto a device used with the introducer sheath.

The lock actuator 802 may be constructed as a lever mechanism with lever portions 802a. The lock actuator 804 can be activated to lock a device used with an introducer sheath and/or the elongated body of the introducer sheath in a secure position using a catch member 806. When the lock actuator 802 is activated, the catch member 806 serves to allow one of the lever portions 802a to be secured by the catch member 806. As shown in FIG. 12B, one of the lever portions 802a may be moved toward the other lever portion 802a (see downward arrow). As the lever portion 802a moves downward, it will be secured by the catch member 806. The catch member 806 includes a barb-like structure that serves to secure the lever portion 802a.

In operation, when the lock actuator 802 is activated, the lock member can be compressed onto the elongated body of an introducer sheath and hold the introducer sheath and/or a device used therewith. The lock member 804 includes an opening such that an introducer sheath and/or a device used therewith may be inserted therethrough. When compressed, the void 808 of the lock member will become smaller or close to allow for reduction of the opening that the introducer sheath and/or device is inserted through. In the example where an introducer sheath is to be secured, the donut structure of lock member 804 includes an inner annular diameter 804a that allows the elongated body of an introducer sheath to be inserted through the lock member 804 before it is compressed. In this configuration, the device lock 800 can be adjusted along the length of an elongated body of an introducer sheath. Suture connections 808 allow a user to secure the device securement 800 to a patient's body, such as by suture tie down.

FIGS. 13-15C illustrate another embodiment of an expander 900. As shown, the expander 900 is a collapsible expander tool that may be carried inside the elongated body of any of the introducer sheaths described herein. It will be appreciated that the expander 900 may be employed as a substitute to any of the previously described expanders. As with the previously described expanders, expander 900 also can be effective to expand a working space inside the body of a patient, while being used with the introducer sheath. The expander 900 also allows the introducer sheath to get sufficiently close to a target area, so as to allow optimal positioning of various tools to be used in performing a desired procedure inside a patient.

In FIG. 13, the expander 900 is self-expanding, collapsible, and constructed of a material utilizing an elastic property. In one example, the expander 900 includes an expanding structure 902 that can be configured as a self-expanding shape memory material, which can also be temporarily collapsed when confined. As shown, the expanding structure 902 is a cage-like structure that is cylindrically shaped and is generally open at its center. In this configuration, the expanding structure 902 can allow various tools inserted through the elongated body to be passed into and through the expanding structure 902 when it is expanded.

In one embodiment, the material of the expanding structure 902 allows the expander 900 to be collapsed on itself, when it is not to be deployed. When the expanding structure 902 is not to be deployed, it can be collapsed into a smaller dimension or diameter by being retracted, for example within the elongated body of an introducer sheath. In operation, the expander 900 can be delivered to a target site such as by exposing the expander from the distal end of the elongated body of an introducer sheath. As one example, the expander 900 can be delivered by using a shaft portion 904.

The shaft portion 904 can be contained, for example within the elongated body of an introducer sheath. Similarly to an elongated body of the introducer sheaths described herein, FIGS. 15A-C show that the shaft portion 904 includes a channel extending longitudinally along the shaft and in communication with the generally open center of the expanding structure. The shaft portion 904 has an outer diameter that is slightly smaller than the inner diameter of an elongated body of an introducer sheath. In this configuration, the shaft portion 904 can be inserted into the sheath and longitudinally movable within the sheath. As the shaft portion 904 includes its own channel, it should not obstruct the use of a medical device to be inserted through the introducer sheath.

The shaft portion 904 is attached to the expanding structure 902 through connectors 906 which facilitate the deployment of the expanding structure 902. It will be appreciated that the connectors may be a portion of the expanding structure 902 and can resemble a last row of struts that connect the expanding structure 902 to the shaft portion 904. The shaft portion 904 and/or the connectors 906 can be moved relative to the elongated body of an introducer sheath to expose or not expose the expanding structure 902. In the expanded configuration, the expanding structure 902 would be exposed by pushing it forward relative to an introducer sheath, or by pulling the introducer sheath back relative to the expander 900. In either configuration, the expanding structure 902 can be exposed to be expanded from a distal end of the elongated body of the introducer sheath. That is, the introducer sheath can act to cover and uncover the expander based on relative movement of the introducer sheath and expander. In the non-expanded configuration, the expanding structure 902 could be collapsed by pulling the expanding structure back inside the introducer sheath through distal end of the elongated body, or could be collapsed by pushing the introducer sheath over the expanding structure 902 to cover it.

In FIG. 13, when the expanding structure 902 is exposed from the introducer sheath, the material of the expanding structure 902 is such that it can be self-deployed to create a working space. That is, due to the expander's propensity to expand when the expanding structure 902 is not contained/retracted inside the introducer sheath, a space inside a patient can be expanded after the expander has been exposed from the introducer sheath. As described, the expander can be put back into the introducer sheath, or covered by the introducer sheath, and also removed from the patient after a procedure has been performed.

In one embodiment, the expanding structure 902 may be a flexible material with an elastic-like quality, and that includes a self-expanding force that can sufficiently open a working space in the body of a patient. In one embodiment, when the expanding structure 902 is exposed, it includes a portion connected to the shaft portion 904. The portion is an outwardly tapering portion that, when expanded, is larger than the outer diameter of the shaft portion 904 and an introducer sheath. The expanding structure 902 also includes a portion distal to the taper portion, and that flattens out or becomes generally a uniform circumferential portion. As best shown in FIGS. 15A-C and 16C-D, the portion distal to the taper portion further includes tips at the distal end. It will be appreciated that the tips are configured so as not to damage tissue of the body of the patient. In some examples, the tips may be a blunted or rounded structure, such as a paddle-like surface.

As one example, the expanding structure 902 may be a nitinol cage-like structure. It will be appreciated that the expander 900 is not limited to the specific structure shown, and may be modified so long as the structure employed maintains the function of being self-expanding when deployed and collapsible when retracted. As some further examples, it will be appreciated that the expanding structure 902 may be other materials, such as elastic resins or plastics. It further will be appreciated that the expanding structure 902 may be constructed as a combination of materials, rather than as one material. For example, the actual expanding portion of the expanding portion may be a nitinol or other shape memory/super elastic metal and/or polymers, while a proximate portion which connects to the shaft portion may be a stainless steel or other plastics. It will be appreciated that the materials employed are suitable for use inside the body of a patient.

Likewise, the shaft portion 904 may be sufficiently flexible or have varied flexibility, as necessary or desired, and so as to be suitable for use with an introducer sheath of varying durometers.

The expander provides key functions in that the expander is retractable and is self-expanding once it is exposed. As the expander is disposed within the introducer sheath, the introducer sheath can be allowed to be placed closer to a target region, so that various tools can be better positioned for a particular procedure to be performed.

As described, the expander 900 can be effective to expand a working space inside the body of a patient. The expander 900 also allows the introducer sheath to get sufficiently close to a target area, so as to allow optimal positioning of various tools to be used in performing a desired procedure inside a patient.

FIG. 14 illustrates a flat, unrolled view of one embodiment of a configuration 902a that may be employed in fabricating the expander of FIG. 13. FIG. 14 shows an example of the laser cutting 902a of the expanding structure. Heat expansion is employed to achieve the final configuration of the expanding structure. The expanding structure of an expander may be configured with a cage-like structure. FIG. 14 shows a configuration 902a of an expanding structure including a reticulate configuration. It will be appreciated that the expanding structures is not limited to a cage-like configuration, and may not be a reticulated or open structure. Rather, the expanding structures may have a closed or solid outer surface. It will be appreciated that the configuration 902a may have other varying configurations, and is not limited to the specific configuration shown, as long as the structure can be self-expanding when deployed and collapsible when not in use.

In some embodiments, the dimensions of structure 902a include a length L of approximately 1.5 inches (right to left directions) and a height H circumference of raw tubing of approximately 1.3 inches (top to bottom direction). It also will be appreciated that the dimensions of the structure 902a may vary as necessary and/or desired.

FIGS. 15A-C illustrate side views of the expander 900 of FIG. 13 in operation with an introducer sheath 908. FIG. 15A shows the expander 900 in a non-expanded configuration and inside an introducer sheath 908. FIG. 15B shows the expanding structure 902 of the expander 900 in a partially expanded configuration and exposed from the sheath 908. The arrow indicates that the sheath 908 may be pulled back relative to the expander 900 and to expose the expander 900. FIG. 15C shows the expander 900 in a fully expanded configuration, where the sheath 908 has been further pulled back relative to the shaft portion 904 and expanding structure 902. See arrow of FIG. 15C.

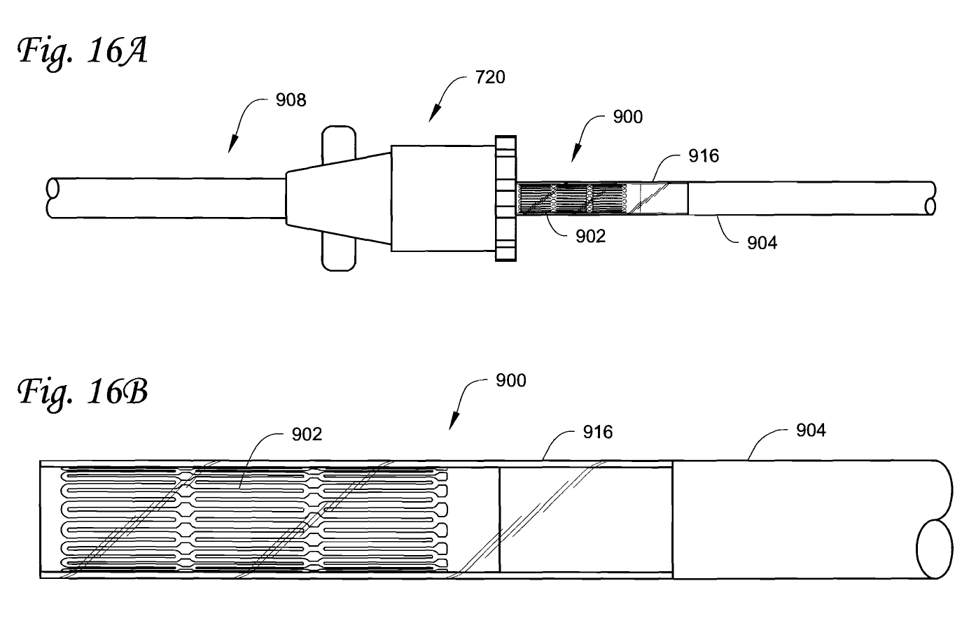

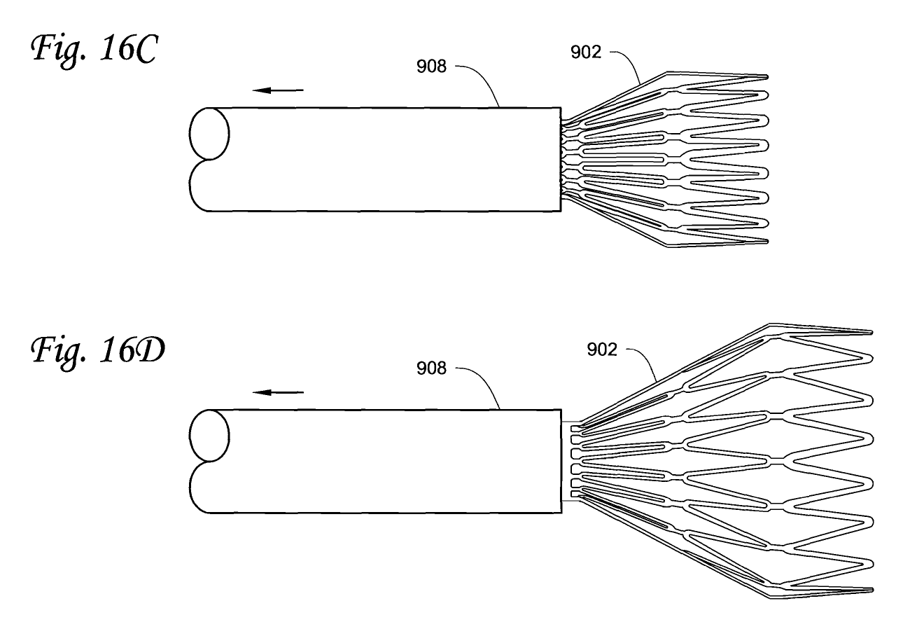

FIGS. 16A-D illustrate the expander 900 in operation with introducer sheath 908 and device lock 720. As described above, the expander 900 includes an expanding structure 902 connected to a shaft portion 904, and is insertable into the introducer sheath 908, such as at the proximate end where the device lock 720 is disposed. The device lock 720 is similar to the device lock 700 described above, and is not further described.

FIG. 16A shows the expander 900 being loaded into an introducer sheath 908 using a loading tool 916. In one embodiment, the loading tool 916 is configured as a cover to contain the expanding structure 902, so the expander 900 can be loaded into the channel of the introducer sheath 908. As one example, the loading tool 916 is a separate sheath, and may be pulled back along the expanding structure 902 and shaft portion 904 as the expander 900 is inserted into the introducer sheath 908. It will be appreciated that the loading tool 916 also may be partially inserted into the sheath 908, so as to help guide the expander 900 into the sheath 908. Once the sheath 908 has been inserted, the loading tool 916 may be discarded or peeled away.

FIG. 16B shows the expander 900 and the loading tool 916. FIGS. 16C-D respectively show the expander 900 in partially expanded and fully expanded configurations. In such configurations, the expander 900 has been inserted into the introducer sheath 908 and is now at the distal end of the introducer sheath 908. In the expanded configurations shown, the introducer sheath 908 has been moved relative to the expander 900 (i.e. as a pull back) to expose the expanding structure 902, such as shown by the arrows in FIGS. 16C-D and also described above in FIGS. 15A-15C.

In yet another embodiment, any of the introducer sheaths described may be provided within an access kit. In one non-limiting example, the access kit includes an introducer sheath, a series of dilators, and an access needle.

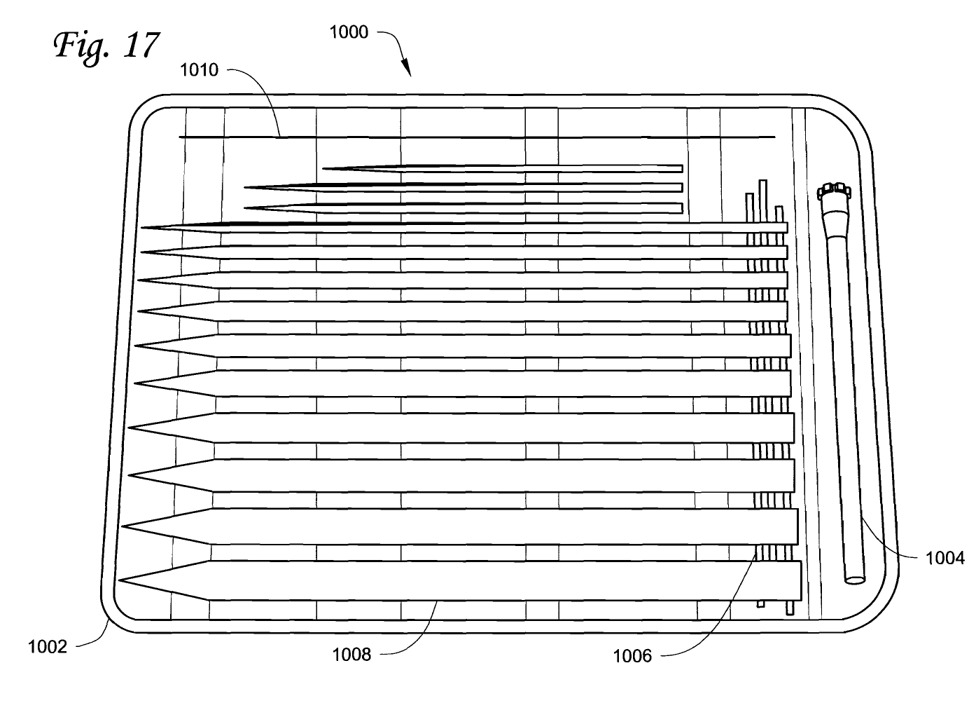

FIG. 17 illustrates one embodiment of an access kit 1000. The access kit includes packaging 1002, a general introducer sheath 1004, a series of dilators 1008, and an access needle 1006. In one embodiment, each dilator is constructed of a flexible material, such as polyethylene, and includes a tapered tip for ease of insertion. Each dilator is configured to allow a guidewire to be inserted therethrough, and has a tapered tip configured to fit about an outside diameter of a guidewire for use to dilate a path to position the dilators at a desired area inside a patient's body where a procedure is to be performed.

In one embodiment, the series of dilators range from 6 Fr. to 30 Fr. and are configured with 2 Fr. increments. It will be appreciated that any of the introducer sheaths described herein are suitable for the kit 1000. As shown, the kit 1000 includes packaging 1002, an introducer sheath 1004, a series of dilators 1008, and a needle 1006. The kit may also include a guidewire 1010. It will be appreciated that guidewires are well known and are commercially available, for example in the use of introducing catheter products in the body of a patient. In one example, a guidewire may have a 0.035 inch diameter.

In one embodiment, the kit 1000 may be configured to not include an expander, such as expander 900. However, the kit is not limited to such a configuration and may include the expander if desirable and/or necessary. It further will be appreciated that the kit is not limited to the specific configuration or components shown in FIG. 17, and may be modified as necessary and/or desired.

In operation, the kit 1000 provides a user with tools to access an area inside the body of a patient. The needle 1006 is used to puncture, open or create the initial incision into a patient's body. The guidewire 1010 is fed through the needle 1006 into the patient and to a desired location where a procedure is to be performed. The needle 1006 can then be removed to allow the use of the series of dilators 1008 passed over the guidewire 1010 to increase the opening into the patient, and to allow for insertion of the introducer sheath 1004. The series of dilators 1008 is used by passing them one by one over the guidewire to increase the opening in diameter increments, and to obtain the size necessary for inserting the introducer sheath 1004. When the opening is large enough, the introducer sheath 1004 can be inserted and secured to its desired position. As one example, once the last dilator (i.e. 30 F dilator) has been positioned, the dilator can be left in place, and the sheath 1004 can be passed over the proximal end of the dilator and pushed into position, where the dilator can then be removed from inside the sheath at the proximal end. In yet another example, once the last dilator has been positioned, the dilator can be removed while the guidewire remains in place. The sheath can then be passed over the dilator (outside the patient). The sheath/dilator combination can then be passed over the guidewire and into position, and then the dilator removed. Subsequent tools as the expander and other medical devices for a particular procedure can then be introduced through the introducer sheath.

The above inventive concepts can be useful in a variety of medical procedures employing introducer sheaths. In one example, the inventive concepts herein can be useful for a sub-xiphoid approach to pass a device and/or medical treatment products to the heart of a patient. In yet another example, the design is particularly useful with a device being used proximate the left atrial appendage of the heart for closing off the same. The introducer sheaths herein can provide easier navigation to the left atrial appendage, thereby providing minimally invasive access, while preventing damage to the pericardium and pericardial sac. Furthermore, the introducer sheaths herein, and any devices and/or medical treatment products introduced by the sheaths, can be suitably secured to a patient. The introducer sheath and the devices and/or medical treatment products introduced by the sheath do not undesirably become dislodged.

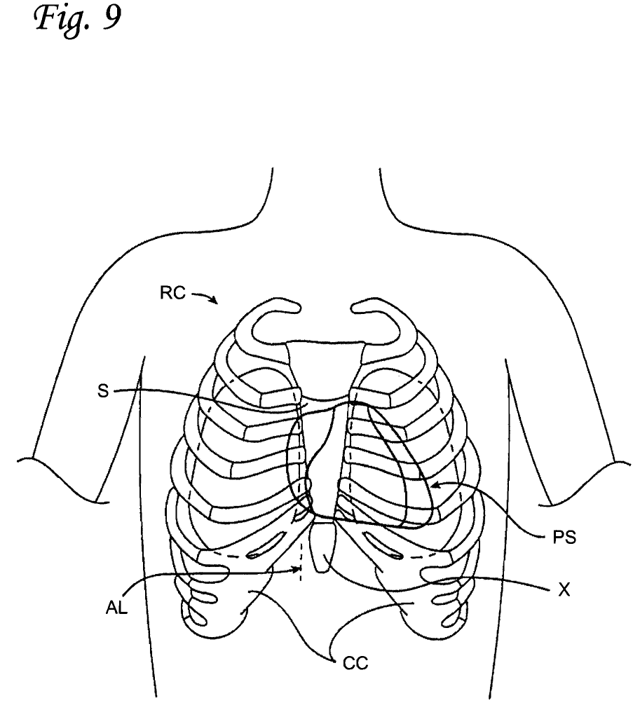

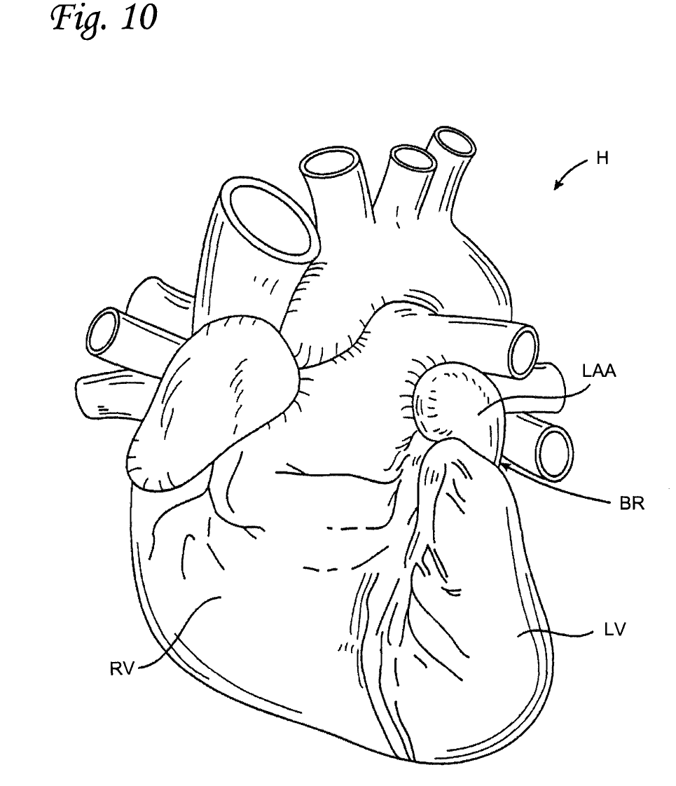

As mentioned above, the introducer sheaths described can be particularly useful for procedures using a subxiphoid access, and that may employ device(s) for procedures on the heart, for instance closing the left atrial appendage. FIGS. 9 and 10 illustrate one example of the environment associated with a subxiphoid approach and associated with the surface of the heart, namely the left atrial appendage. The subxiphoid approach and environments associated therein, including access to the heart, is described in U.S. Pat. No. 6,488,689, which is herein incorporated by reference in its entirety.

Referring now to FIG. 9, the heart is located within the pericardial space PS located beneath the patient's rib cage RC. The sternum S is located in the center of the rib cage RC and terminates at its lower end in the xiphoid X. On either side of the xiphoid are the costal cartilage CC, while showing percutaneous access points for performing sub-xiphoid procedures, which are located beneath the rib cage RC, and may be between the xiphoid X and an adjacent costal cartilage CC. One example of such a percutaneous access point is the access location AL shown by a broken line.

Through the subxiphoid approach, access can be achieved to various locations inside body, for example the heart. FIG. 10 is an anterior view of a heart H illustrating the right ventricle RV, the left ventricle LV, and the left atrial appendage LAA. It will be appreciated that one desire of the introducer sheath disclosure herein is to introduce a closure structure/device that can close off the base region BR of the left atrial appendage LAA. By closing off the base region BR, the exchange of materials between the left atrial appendage LAA and the left atrium LA is stopped. For example, the release of emboli from the left atrial appendage LAA into the left atrium can be stopped. It will be appreciated that the introducer sheaths described herein may be employed in various applications and procedures other than for accessing the heart and for closing a left atrial appendage, and may be modified as suitable and/or necessary.

The invention may be embodied in other forms without departing from the spirit or novel characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

1. An introducer sheath comprising:
an elongated body including a proximate end and a distal end, the ends are oppositely disposed and each end including an opening, the elongated body having a wall structure with a channel extending longitudinally through the elongated body and the openings, the elongated body being decreasingly stiff along its longitudinal profile from the proximate end to the distal end, such that the introducer sheath having a decreasing stiffness from the proximate end to the distal end.

2. The introducer sheath of claim 1, wherein the elongated body is generally flexible and comprises at least one lumen extending longitudinally through the elongated body, the at least one lumen is disposed within the wall structure of the elongated body and outside the periphery of the channel,
the introducer sheath further comprising at least one support member insertable into the lumen, the at least one support member having a varying stiffness such that the introducer sheath having a decreasing stiffness from the proximate end to the distal end when the at least one support member is inserted into the at least one lumen.

3. The introducer sheath of claim 1, wherein the wall structure of the elongated body having a decreasing stiffness from the proximate end to the distal end.

4. The introducer sheath of claim 1, wherein the elongated body comprising a plurality of zones having differing stiffness, where a first zone including the proximate end is more stiff than a zone between the proximate end and the distal end, an a zone including the distal end is the least stiff.

5. The introducer sheath of claim 1, wherein the elongated by comprising an outer surface having a tapered surface, where the distal end has a thinner profile than the proximate end.

6. The introducer sheath of claim 1, further comprising a retention mechanism disposed proximate one end of the elongated body, the retention mechanism is adjustable along the elongated body, such that the elongated longitudinal body is configured to be insertable into a patient at varying lengths, the retention mechanism is configured to retain the elongated body in place after a portion of the elongated body is positioned inside the body of a patient, the retention mechanism is configured to prevent the elongated longitudinal body from being dislodged.

7. The introducer sheath of claim 6, wherein the retention mechanism comprising a stop component connected to the elongated body, the stop component is configured to prevent inward movement of the elongated body, and another stop component connected to the elongated body, the another stop component is configured to prevent outward movement of the elongated body.

8. The introducer sheath of claim 7, wherein the stop component including an increased diameter toward the proximate end, and the another stop component is an expandable member.

9. The introducer sheath of claim 6, wherein the retention mechanism comprising a stabilizing member and a plurality of connecting members connected to the stabilizing member, the stabilizing member is longitudinally adjustable along the elongated body, and is configured to be secured to the elongated body, the connecting members are configured to secure the stabilizing member to a patient.

10. The introducer sheath of claim 1, further comprising a device lock, the device lock is disposed at the proximate end of the elongated body, the device lock includes a lock member that is configured to secure a device introduced through the elongated body.

11. The introducer sheath of claim 10, wherein the lock member that is compressible.

12. The introducer sheath of claim 11, wherein the device lock comprising a cap and a body configured to rotate relative to each other, such that relative rotation between the cap and the body compresses the lock member.

13. The introducer sheath of claim 11, wherein the device lock comprising a lever mechanism, such that movement of the lever compresses the lock member.

14. The introducer sheath of claim 1, wherein the distal end comprises a distal tip being rounded and including a lubricious coating thereon.

15. The introducer sheath of claim 1, further comprising an expander disposed proximate the distal end of the elongated body, the expander is configured to expand and create a working space inside a targeted anatomy.

16. An introducer sheath for minimally invasive medical procedures comprising:
an elongated body including a proximate end and a distal end, the ends are oppositely disposed and each end including an opening, the elongated body having a channel extending longitudinally through the elongated body and the openings;
an expander disposed proximate the distal end of the elongated body, the expander is configured to expand and create a working space inside a targeted anatomy of a patient, the expander comprising an expanding structure connected to a shaft portion, the expanding structure is generally open at a center thereof and the shaft portion includes a channel extending longitudinally therethrough and in communication with the center of the expanding structure, the expander is insertable through the channel of the elongated body, the expanding structure is self-expanding is configured to expand when extended out of the channel at the distal end and exposed, and the expanding structure is collapsible when retracted into the channel of the distal end;
a device lock disposed at the proximate end of the elongated body, the device lock includes a lock member that is compressible, and the lock member is configured to secure a device introduced through the channel of the elongated body and the channel of the shaft portion.

17. The introducer sheath of claim 16, wherein the expanding structure is a cage-like structure and composed of a shape memory and elastic material.

18. The introducer sheath of claim 16, further comprising a loading tool configured to contain the expanding structure from expanding and allow the expander to be inserted into the channel of the elongated body.

19. A kit for introducing medical devices employed within a body of a patient comprising:
an access needle;
a guidewire insertable through the needle;
a plurality of dilators insertable into a body of a patient, each dilator having a tapered tip and being configured to be passed over the guidewire;
an introducer sheath comprising
an elongated body including a proximate end and a distal end, the ends are oppositely disposed and each end including an opening, the elongated body having a channel extending longitudinally through the elongated body and the openings, and
a device lock disposed at the proximate end of the elongated body, the device lock includes a lock member that is compressible, the lock member is configured to secure a device introduced through the channel of the elongated body; and
packaging to contain the access needle, dilators, guidewire, and introducer sheath.

20. The kit of claim 19, further comprising an expander disposed proximate the distal end of the elongated body, the expander is configured to expand and create a working space inside a targeted anatomy of a patient, the expander comprising an expanding structure connected to a shaft portion, the expanding structure is generally open at a center thereof and the shaft portion includes a channel extending longitudinally therethrough and in communication with the center of the expanding structure, the expander is insertable through the channel of the elongated body, the expanding structure is self-expanding is configured to expand when extended out of the channel at the distal end and exposed, and the expanding structure is collapsible when retracted into the channel of the distal end;
